(19) United States
(12) Patent Application Publication
(10) Pub. No.: US 2003/0082117 A1
(43) Pub. Date: May 1, 2003

(54) Genus leontopodium plant extract and compositions containing same

(76) Inventors: Richard Martin, Rochecorbon (FR); Beatrice Belcour, Joinville Le Pont (FR); Pascal Hilaire, Vouvray (FR); Roger Rozot, Lagny-sur-Marne (FR)

Correspondence Address: BURNS DOANE SWECKER & MATHIS L L P, POST OFFICE BOX 1404, ALEXANDRIA, VA, 22313-1404, US

(21) Appl. No.: 10/168,026

(22) PCT Filed: Dec. 14, 2000
(86) PCT No.: PCT/FR00/03529

(30) Foreign Application Priority Data
Dec. 23, 1999 (FR) 99/16379

(51) Int. Cl. A61K 35/78; A61K 7/42

(52) U.S. Cl. 424/59; 424/725

(57) ABSTRACT

The invention concerns in its most general aspect an extract of dedifferentiated cells of a least a genus Leontopodium plant, an ultraviolet radiation filtering agent consisting of at least an extract of dedifferentiated cells of at least a genus Leontopodium plant, a composition containing at least an extract of dedifferentiated cells of a genus Leontopodium plant, the use of said extract as ultraviolet radiation filtering agent and a cosmetic method using a composition comprising said extract.

The present invention relates, in its most general terms, to an extract of dedifferentiated cells of at least one plant of the genus Leontopodium, to an agent for screening out ultraviolet radiation consisting of at least one extract of dedifferentiated cells of at least one plant of the genus Leontopodium, to a composition containing at least one extract of dedifferentiated cells of at least one plant of the genus Leontopodium, to the use of said extract as an agent for screening out ultraviolet radiation, and to a cosmetic treatment method involving a composition comprising said extract.

Extracts of plants of the genus Leontopodium are provided in the prior art as an agent for protecting against ultraviolet radiation. Moreover, the company Alban Muller International (France) markets such an extract. According to the supplier, it is an extract obtained, using a solvent composed of water and propylene glycol, from leaves and flowers of Edelweiss plants grown in vivo (high mountain region).

The very well-known Edelweiss belongs to the plant genus Leontopodium. Plants of the genus Leontopodium are rare plants which grow spontaneously at altitudes exceeding 2 000 meters, particularly in the mountains of the Alps and/or of Nepal. By virtue of their rarity, these plants are protected and the gathering thereof is the subject of extremely strict regulations. This, added to the fact that the natural conditions for growing these plants are particular, means that growing them industrially proves to be difficult, to say the least, under conditions which both do not endanger the species used and maintain reasonable exploitation costs.

Culturing dedifferentiated cells of plants of the genus Leontopodium therefore appears to be a solution to the problems set out above, all the more so since this introduces, among other advantages, the fact that the plant material which can be used is no longer seasonal but accessible throughout the year and in all weathers, thus satisfying the quantitative criteria of industry. In addition, such cultures are perfectly reproducible since they are grown under perfectly controlled conditions of temperature, of pH and of culture medium, and satisfy industrial criteria of quality.

The term “dedifferentiated plant cells” is intended to mean any plant cell which exhibits no characteristic of a particular specialization and is capable of living by itself and not in a state of dependency on other cells. These dedifferentiated plant cells are optionally capable, under the effect of an induction, of any differentiation in accordance with their genome. Depending on the culture method chosen, and in particular depending on the culture medium chosen, it is possible to obtain, from the same explant, dedifferentiated plant cells which have different characteristics. Specifically, one of the originalities of plant cells obtained in vitro is the very great variability which is spontaneously established in the cultures. This variability is expressed in terms of the physiology and biochemistry. De facto, the culturing in vitro leads to the rupture of the cellular networks which exist in the tissues in vivo. Gradually, in the course of the subculturing, new networks of interactions between these cells cultured in vitro and their new environment are randomly established. These new interactions are sources of new metabolites and/or of modulations of expression of metabolites present in the plant.

However, nothing in the prior art indicates that extracts of dedifferentiated plant cells will conserve the properties of the extracts of plant cells obtained from plants grown in vivo. Particularly, regarding plants of the genus Leontopodium, there is nothing to indicate or suggest that the screening properties developed naturally by a natural plant exposed, due to its biotope, to large amounts of ultraviolet radiation will be conserved by dedifferentiated cells which, because of the conditions under which they are cultured, are not exposed to large amounts of ultraviolet radiation and which, by nature, have lost the characteristics of particular specialization of the natural cells.

The term “culturing in vivo” is intended to mean any culturing of the conventional type, i.e. in the ground, in the open air or under glass, or alternatively out of the ground.

The term “culturing in vitro” is intended to mean all of the techniques known to those skilled in the art which make it possible artificially to obtain a plant or a part of a plant. The selection pressure imposed by the physicochemical conditions during the growth of the plant cells in vitro makes it possible to obtain a standardized plant material which is available throughout the year, unlike plants grown in vivo.

The Applicant has now shown that it is possible to obtain extracts of dedifferentiated cells of at least one plant of the genus Leontopodium, and that such extracts have properties of screening out ultraviolet radiation which make them a product of choice which can be used as an agent for screening out ultraviolet radiation.

A first subject of the invention is therefore, as a novel product, an extract of dedifferentiated cells of at least one plant of the genus Leontopodium.

The genus Leontopodium belongs to the Compositae family which has more than 1 300 genera and 21 000 species. The genus Leontopodium comprises approximately 10 species, among which mention may be made of Leontopodium alpinum, Leontopodium stracheyi, Leontopodium nivale, Leontopodium palibinianum (or Leontopodium sibiricum), Leontopodium souliei, Leontopodium linearifolium and Leontopodium gnaphalioides.

Thus, the extract of dedifferentiated cells of at least one plant of the genus Leontopodium of the invention is an extract prepared from dedifferentiated cells derived from at least one plant belonging to a species chosen from Leontopodium alpinum, Leontopodium stracheyi, Leontopodium nivale, Leontopodium palibinianum (or Leontopodium sibiricum), Leontopodium souliei, Leontopodium linearifolium and Leontopodium gnaphalioides.

Preferentially according to the invention, the extract is prepared from dedifferentiated cells derived from a plant belonging to the species Leontopodium alpinum or to the species Leontopodium stracheyi.

Besides the fact that the extract of dedifferentiated cells of at least one plant of the genus Leontopodium has properties of screening out ultraviolet radiation, it has, among advantages other than those described above for the those dedifferentiated, that of having a light absorption spectrum strictly limited to UV radiation, with no absorption of visible radiation. This is a not insignificant advantage compared to an extract obtained with a whole plant which has an absorption in the visible range, leading to coloration of the extract, making it difficult to use directly, particularly in cosmetics. The extract of dedifferentiated cells of at least one plant of the genus Leontopodium may be used directly without any decoloration steps which, in general, prove to be long and expensive for an incomplete result, the whole plant extract not generally being totally decolored.

In the remainder of the text, the term “dedifferentiated cells of Leontopodium” should be understood as “dedifferentiated cells of at least one plant of the genus Leontopodium”. Similarly, the term “extract of Leontopodium” should be understood as “extract of dedifferentiated cells of Leontopodium.” and therefore as “extract of dedifferentiated cells of at least one plant of the genus Leontopodium”.

The extract of dedifferentiated cells of at least one plant of the genus Leontopodium may be prepared by any extraction method known to those skilled in the art. Mention may, in particular, be made of alcoholic extracts, especially ethanol extracts and or methanol extracts, aqueous-alcoholic extracts or aqueous extracts, such as those for which the method of preparation is described in French patent No. 95-02379.

According to the invention, an ethanol extract is preferentially used.

Whatever the method for preparing the extract, the latter may then be lyophilized for optional storage.

An example of preparing an extract which can be used according to the invention is given elsewhere in the examples.

Solar radiation is composed, inter alia, of ultraviolet radiation type A which has a wavelength of between 320 nm and 400 nm (UV-A), of ultraviolet radiation type B which has a wavelength of between 280 and 320 nm (UV-B) and of ultraviolet radiation type C which has a wavelength of between 200 and 280 nm (UV-C). UV-Bs are highly energetic and relatively nonpenetrating, make up a small proportion of sunlight and are dependent on climatic variations (weather cloudy, overcast, etc.), and their presence varies according to the time of day (notion of peak (zenith)). UV-As are less energetic than UV-Bs but more penetrating, are present in great amounts in sunlight (a minimum of 100 times more UV-A than UV-B), are relatively independent of climatic variations and are present whatever the time of day. UV-Cs are highly energetic and relatively nonpenetrating. They are theoretically stopped by the ozone layer. However, potentially, they may be responsible for nucleic acid damage.

It is known that solar radiation is responsible for beneficial effects on the skin, such as for example darkening, but it is also responsible for inducing damage to the skin, in particular in the case of “sensitive” skin or of skin which is continually exposed.

In terms of benefits, darkening, commonly called tanning, is an essential element of the defense system of the skin. Specifically, in response to ultraviolet radiation, the melanocytes of the upper layer of the epidermis synthesize melanin which, once incorporated into the keratinocytes, constitutes a screen located at the surface of the skin, which screen absorbs ultraviolet radiation. The objective of this is to decrease the amount of ultraviolet radiation which crosses the layers of the skin, in order to prevent it reaching the deep layers and causing damage there which is harmful to the skin.

In terms of harmful effects, it is known that excessive exposure of the skin to ultraviolet radiation, to solar radiation in particular, may lead to a change in the elasticity of the skin and in its content of certain compounds, and thus promote acceleration of the natural process of aging of the skin. This process of accelerated or premature aging due to ultraviolet radiation is generally called photo-aging or actinic aging or dermatoheliosis. Weakly penetrating UV-Bs reach mainly the epidermis. The role of UV-Bs has been clearly demonstrated in the induction of UV-induced skin cancers. Their principal chromophore is, in fact, nucleic acids, in particular deoxyribonucleic acid, in which they induce damage and/or mutations (Eller M. S., 1995, in Photodamage, 26-56, Blackwell ed.).

Now, plants of the genus Leontopodium, because of the altitude at which they grow, are highly exposed to ultraviolet radiation. In fact, at the risk of seeing them disappear, they must have systems for protection against damage induced by ultraviolet radiation.

Here again, after long studies, the Applicant has shown that an extract of dedifferentiated cells of at least one plant of the genus Leontopodium, as defined above, has properties of screening out ultraviolet radiation.

According to the invention, the expression “screening out ultraviolet radiation” is intended to mean the power to partially or totally stop ultraviolet radiation type A and/or B and/or C.

Thus, a second subject of the invention is an agent for screening out ultraviolet radiation, characterized in that it consists of at least one extract of dedifferentiated cells of at least one plant of the genus Leontopodium, as defined above.

In addition, the Applicant has shown that an extract obtained with dedifferentiated cells of a plant of the genus Leontopodium alpinum has an absorption spectrum which covers the entire ultraviolet spectrum (UV-A, UV-B and UV-C).

Thus, a third subject of the invention is an agent for screening out ultraviolet radiation type A, B and C, characterized in that it consists of at least one extract of dedifferentiated cells of at least one plant. of the species Leontopodium alpinum.

The Applicant has also shown that an extract obtained with dedifferentiated cells of a plant of the genus Leontopodium stracheyi has an absorption spectrum which covers specifically only the part of the spectrum corresponding to ultraviolet radiation type B and C.

Thus, a fourth subject of the invention is an agent for screening out ultraviolet radiation type B and C, characterized in that it consists of at least one extract of dedifferentiated cells of at least one plant of the species Leontopodium stracheyi.

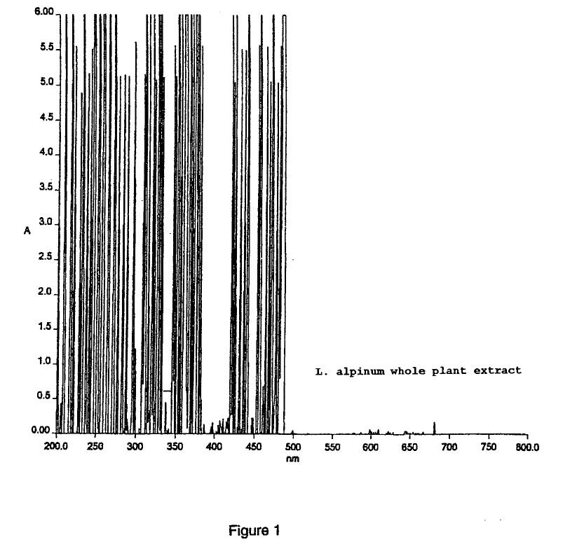

FIG. 1 shows a spectrum of ultraviolet radiation absorption by an extract of the invention made from a whole plant using a plant of the genus Leontopodium alpinum.

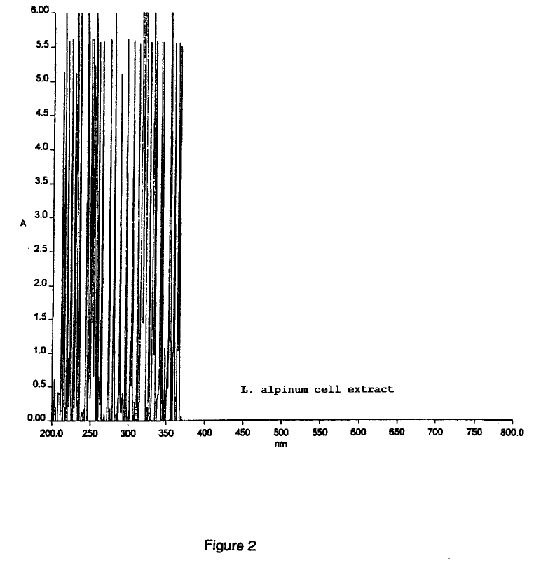

FIG. 2 shows a spectrum of ultraviolet radiation absorption by an extract of the invention made from dedifferentiated cells using a plant of the genus Leontopodium alpinum.

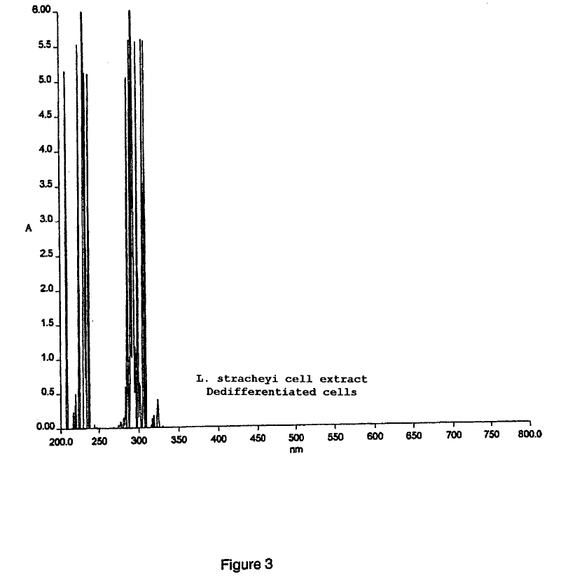

FIG. 3 shows a spectrum of ultraviolet radiation absorption by an extract of the invention made from dedifferentiated cells using a plant of the genus Leontopodium stracheyi.

A fifth subject of the invention is a composition comprising, in a physiologically acceptable medium, at least one extract of dedifferentiated cells of the genus Leontopodium, as defined above.

A sixth subject of the invention is a composition comprising, in a physiologically acceptable medium, at least one agent for screening out ultra-violet radiation, characterized in that it consists of at least one extract of dedifferentiated cells of at least one plant of the genus Leontopodium, as defined above.

A seventh subject of the invention is a composition comprising, in a physiologically acceptable medium, at least one agent for screening out ultra-violet radiation type A, B and C, characterized in that it consists of at least one extract of dedifferentiated cells of at least one plant of the species Leontopodium alpinum.

A seventh subject of the invention is a composition comprising, in a physiologically acceptable medium, at least one agent for screening out ultra-violet radiation type B and C, characterized in that it consists of at least one extract of dedifferentiated cells of at least one plant of the species Leontopodium stracheyi.

The expression “physiologically acceptable medium” is understood to mean a medium compatible with the skin, mucous membranes, nails and hair.

Whatever its form, the composition of the invention may be a cosmetic or dermatological composition. According to the invention, the composition is preferentially a cosmetic composition.

Whatever the form of the composition according to the invention, the amount of extract of at least one plant of the genus Leontopodium contained in the composition depends, of course, on the desired effect and may therefore vary within a wide range.

To give an order of magnitude, the amount of extract of at least one plant of the genus Leontopodium contained in the composition is an amount representing from 0.001% to 20% of the total weight of the composition, and preferentially an amount representing from 1% to 10% of the total weight of the composition.

Of course, the composition of the invention may, in addition to the screening agent consisting of at least one extract of dedifferentiated cells of at least one plant of the genus Leontopodium as defined above, comprise at least one other compound recognized as an agent for screening out ultraviolet radiation.

Thus, an eighth subject of the invention is a screening composition, characterized in that it comprises, in a physiologically acceptable medium, an extract of dedifferentiated cells of at least one plant of the genus Leontopodium and at least one other compound recognized as an agent for screening out ultraviolet radiation.

The extract of dedifferentiated cells of at least one plant of the genus Leontopodium is an extract as described previously in the text.

The other compound recognized as an agent for screening out ultraviolet radiation is preferably chosen from organic screening agents and/or inorganic screening agents.

Organic screening agents which may be mentioned include, in particular, cinnamic derivatives, salicylic derivatives, camphor derivatives, triazine derivatives, benzophenone derivatives, dibenzoylmethane derivatives, β,β-diphenylacrylate derivatives, p-aminobenzoic acid derivatives, the screening polymers and screening silicones described in application WO-93/04665 or the organic screening agents described in patent application EP-A 0 487 404.

Inorganic screening agents which may be mentioned include, in particular, pigments or alternatively nanopigments (mean size of the primary particles: generally between 5 nm and 100 nm, preferably between 10 and 50 nm) of metal oxides, which may or may not be coated, such as for example nano-pigments of titanium oxide (amorphous or crystallized in rutile and/or anatase form), iron oxide, zinc oxide, zirconium oxide or cerium oxide, which are all photo-protective agents well known per se which act by physically blocking (reflecting and/or diffusing) UV radiation. Conventional coating agents are; moreover, alumina and/or aluminum stearate. Such coated or uncoated metal oxide nanopigments are in particular described in patent applications EP-A-0 518 772 and EP-A-0 518 773.

Examples of additional sunscreens active in UV-A and/or UV-B which may be mentioned include:

p-aminobenzoic acid,

oxyethylenated (25 mol) p-aminobenzoate,

2-ethylhexyl p-dimethylaminobenzoate,

N-oxypropylenated ethyl p-aminobenzoate,

glyceryl p-aminobenzoate,

homomenthyl salicylate,

2-ethylhexyl salicylate,

triethanolamine salicylate,

4-isopropylbenzyl salicylate,

4-tert-butyl-4′-methoxydibenzoylmethane,

4-isopropyldibenzoylmethane,

menthyl anthranilate,

2-ethylhexyl 2-cyano-3,3′-diphenylacrylate,

ethyl 2-cyano-3,3′-diphenylacrylate

2-phenylbenzimidazole-5-sulfonic acid and salts thereof,

3-(4′-trimethylammonium)benzylidenebornan-2-one methyl sulfate,

2-hydroxy-4-methoxybenzophenone,

2-hydroxy-4-methoxybenzophenone-5-sulfonate,

2,4-dihydroxybenzophenone,

2,2′,4,4′-tetrahydroxybenzophenone,

2,2′-dihydroxy-4,4′-dimethoxybenzophenone,

2-hydroxy-4-n-octoxybenzophenone,

2-hydroxy-4-methoxy-4′-methylbenzophenone,

α-(2-oxoborn-3-ylidene),tolyl-4-sulfonic acid and salts thereof,

3-(4′-sulfo)benzylidenebornan-2-one and salts thereof,

3-(4′-methylbenzylidene)-d,1-camphor,

3-benzylidene-d,1-camphor,

benzene-1,4-bis(3-methylidene-10-camphosulfonic acid) and salts thereof,

urocanic acid,

2,4,6-tris[p-(2′-ethylhexyl-1′-oxycarbonyl)anilino]-1,3,5-triazine,

2-[(p-(tertbutylamido)anilino]-4,6-bis[(p-(2′-ethyl-hexyl-1′-oxycarbonyl)anilino]-1,3,5-triazine,

2,4-bis{[4-(2-ethylhexyloxy)]-2-hydroxyphenyl}-6-(4-methoxyphenyl)-1,3,5-triazine,

the polymer of N-(2 and 4)-[(2-oxoborn-3-ylidene)-methyl]benzyl]acrylamide,

4,4-bis(benzimidazolylphenylene)-3,3′,5,5′-tetra-sulfonic acid and salts thereof,

2,2′-methylenebis[6-(2H-benzotriazol-2-yl)-4-(1,1,3,3-tetramethylbutyl)phenol],

polyorganosiloxanes containing a malonate function.

The amount of compound recognized as an agent for screening out ultraviolet radiation, contained in the composition of the invention, depends, of course, on the desired effect and may therefore vary within a wide range.

To give an order of magnitude, the amount of agent for screening out ultraviolet radiation, other than the extract of at least one plant of the genus Leontopodium, contained in the composition is an amount representing from 0.1% to 20% of the total weight of the composition, and preferentially an amount representing from 0.5% to 10% of the total weight of the composition.

The compositions according to the invention may also contain agents for artificially tanning and/or darkening the skin (self-tanning agents), such as, for example, dihydroxyacetone (DHA).

The compositions of the invention may also comprise conventional cosmetic adjuvants, in particular chosen from fatty substances, organic solvents, thickeners, softeners, antioxidants, opacifiers, stabilizers, emollients, hydroxy acids, antifoaming agents, moisturizers, vitamins, fragrances, preserving agents, surfactants, fillers, sequestering agents, propellants, acidifying or basifying agents, dyes, or any other ingredient conventionally used in cosmetics, in particular for manufacturing antisun compositions in the form of emulsions.

The fatty substances may consist of an oil or a wax or mixtures thereof, and they also comprise fatty, acids, fatty alcohols and fatty acid esters. The oils may be chosen from animal, plant, mineral and synthetic oils, and in particular from liquid petroleum jelly, liquid paraffin, volatile or non-volatile silicone oils, isoparaffins, fluoro oils and perfluoro oils. Similarly, the waxes may be chosen from animal, fossil, plant, mineral or synthetic waxes which are known per se.

Among the organic solvents which may be mentioned are lower alcohols and polyols.

The thickeners may in particular be chosen from crosslinked homopolymers of acrylic acid, and guar gums and celluloses which may or may not be modified, such as hydroxypropylated guar gum, methylhydroxyethyl-cellulose, hydroxypropylmethylcellulose or hydroxy-ethylcellulose.

Of course, those skilled in the art will take care to select this or these optional additional compounds and/or the amounts thereof such that the advantageous properties, in particular the level of photoprotection, intrinsically associated with the binary combination in accordance with the invention are not, or are not substantially, adversely affected by the addition(s) envisioned.

The compositions of the invention may be prepared according to techniques well known to those skilled in the art, in particular those intended for preparing emulsions of oil-in-water or water-in-oil type, or anhydrous compositions.

This composition may, in particular, be in the form of a simple or, complex (O/W, W/O, O/W/O or W/O/W) emulsion such as a cream, a milk, a gel or a cream-gel, of a powder, of a solid composition or of supple pastes, and may optionally be packaged in an aerosol and may be in the form of a mousse or a spray.

When it is an emulsion, the aqueous phase thereof may comprise a nonionic vesicular dispersion prepared according to known processes (Bangham, Standish and Watkins, J. Mol. Biol. 13, 238 (1965), FR2315991 and FR2416008).

The cosmetic composition of the invention may be used as a composition for protecting the human epidermis or the hair against ultraviolet rays, as an antisun composition or as a make-up product.

When the cosmetic composition according to the invention is used for protecting the human epidermis against UV rays, or as an antisun composition, it may be in the form of a suspension or a dispersion in solvents or fatty substances, in the form of a nonionic vesicular dispersion or in the form of an emulsion, preferably of oil-in-water type, such as a cream or a milk, or in the form of an ointment, a gel, a cream-gel, a stick, supple pastes, an aerosol mousse or a spray.

When the cosmetic composition according to the invention is used for protecting the hair, it may be in the form of a shampoo, a lotion, a gel, an emulsion or a nonionic vesicular dispersion and may constitute, for example, a rinse-out composition, to be applied before or after shampooing, before or after dyeing or bleaching, or before, during or after permanent-waving or relaxing the hair, a styling or treating lotion or gel, a blow-drying or hair-setting lotion or gel, or a permanent-waving, relaxing, dyeing or bleaching composition for the hair.

When the composition is used as a make-up product for the eyelashes, the eyebrows or the skin, such as an epidermal treatment cream, a foundation, a stick of lipstick, an eyeshadow, a face powder, a mascara or an eyeliner, it may be in solid or pasty, anhydrous or aqueous form, for instance oil-in-water or water-in-oil emulsions, nonionic vesicular dispersions or suspensions.

By way of indication, for the antisun formulations in accordance with the invention which have a support of the oil-in-water type, the aqueous phase (in particular comprising the hydrophilic screening agents) generally represents from 50 to 95% by weight, preferably from 70 to 90% by weight, relative to the total weight of the formulation, the oily phase (in particular comprising lipophilic screening agents) generally represents from 5 to 50% by weight, preferably from 10 to 30% by weight, relative to the total weight of the formulation, and the (co)emulsifier(s) generally represent(s) from 0.5 to 20% by weight, preferably from 2 to 10% by weight, relative to the total weight of the formulation.

A ninth subject of the invention is also the use, in a composition or for preparing a composition, in a physiologically acceptable medium, of at least one extract of dedifferentiated cells of at least one plant of the genus Leontopodium, as defined above, the extract or the composition being intended to screen out ultraviolet radiation.

A tenth subject of the invention is also the use, in a composition or for preparing a composition intended to screen out ultraviolet radiation, in a physiologically acceptable medium, of a screening agent as defined above.

Preferentially, these compositions are used for protecting the skin and/or the hair against ultraviolet radiation, in particular solar radiation.

A subject of the invention is also a method for the cosmetic treatment of the skin or the hair, intended to protect them against the effects of UV rays, consisting in applying thereto an effective amount of a cosmetic composition as defined above.

The following examples and compositions illustrate the invention without limiting it in any way. In the compositions, the proportions indicated are percentages by weight.

### EXAMPLE 1

### Preparation of Dedifferentiated Cells of Leontopodium stracheyi

Leontopodium stracheyi leaves are removed and decontaminated with saturated solutions of sodium hypochlorite or of calcium hypochlorite at room temperature for several minutes. The tissues are rinsed with sterile distilled water and then placed in a solution of ethanol diluted to 20%. They undergo 3 washes with sterile distilled water at the end of the decontamination. The leaves are then cut up sterilely under a laminar flow hood and then placed in a Petri dish on Murashige & Skoog nutrient agar medium, the composition of which per liter is:

| | | |
|---|---|---|
| Skoog macroelements | 100.0 | ml |
| Skoog microelements | 1.0 | ml |
| Skoog vitamins | 2.0 | ml |
| Iron EDTA | 10.0 | ml |
| 10⁻⁴ M 2,4-D* | 10.0 | ml |
| 10⁻⁴ M kinetin | 0.6 | ml |
| Sucrose | 30.0 | g |
| Agar | 8.0 | g |
| Distilled water | qs 1 | liter |
| pH before sterilization | 5.8 | UpH |

*10⁻⁴ M 2,4-dichlorophenoxyacetic acid (or 10⁻⁴ M 2,4-D)

The composition of the Skoog macroelements is, per liter:

| | | |
|---|---|---|
| KNO₃ | 1 900 | mg |
| NH₄NO₃ | 1 650 | mg |
| MgSO₄.7H₂O | 370 | mg |
| CaCl₂.2H₂O | 440 | mg |
| KH₂PO₄ | 170 | mg |

The composition of the Skoog microelements is, per liter:

| | | |
|---|---|---|
| CuSO₄.5H₂O | 0.025 | mg |
| MNSO₄.1H₂O | 16.900 | mg |
| KI | 0.830 | mg |
| Na₂MoO₄.2H₂O | 0.250 | mg |
| ZnSO₄.7H₂O | 10.600 | mg |
| H₃BO₃ | 6.200 | mg |
| CoCl₂.6H₂O | 0.025 | mg |

The composition of the Skoog vitamins is, per liter:

| | | |
|---|---|---|
| Myoiniositol | 100.0 | mg |
| Nicotinic acid | 0.5 | mg |
| Pyridoxine | 0.5 | mg |
| Thiamine | 0.1 | mg |
| FeSO₄.7H₂O | 27.8 | mg |
| Na₂ EDTA | 37.3 | mg |

The primary calluses appear between 2 and 5 weeks. In the course of the subculturing, the cells stabilize (appearance, color, etc.) and are transferred, in the same liquid culture medium (without agar) into a fementer; The cells obtained in liquid medium are recovered by filtration on a 50 to 100 μm gauze, depending on the size of the cell aggregates. These cells are placed at −20° C. under slow freezing, which promotes the formation of large intracellular crystals allowing the cells to be broken.

### EXAMPLE 2

### Preparation of Dedifferentiated Cells of Leontopodium alpinum

Dedifferentiated cells of Leontopodium alpinum were obtained from Leontopodium alpinum leaves according to the same protocol as that used in example 1.

### EXAMPLE 3

### Preparation of Ethanol Extracts

Extract A: 1.85 g fresh weight of frozen Leontopodium alpinum leaves are ground (mortar, Potter, etc.) in 18.5 ml of ethanol;

Extract B: 2.2 g fresh weight of frozen Leontopodium alpinum calluses are ground (mortar, Potter, etc.) in 22 ml of ethanol;

Extract C: 2.2 g fresh weight of Leontopodium stracheyi calluses are ground (mortar, Potter, etc.) in 22 ml of ethanol.

Each extract is filtered over Prat Dumas No. 5 filter paper in order to remove the aggregates in suspension. Each extract is dried under vacuum (50 mbar/50° C.) and then taken up in 4.4 ml of ethanol (Leontopodium alpinum calluses and Leontopodium stracheyi calluses) or 3.7 ml of ethanol for the Leontopodium alpinum leaves, i.e. approximately twice the mass of plant material in volume of ethanol. This operation makes it possible to remove, inter alia, the polysaccharides extracted in the aqueous phase introduced by the fresh cells. The whole plant extract is a vivid pure green, which is not the case of the cell extracts derived from calluses. This characteristic of the whole plant extract constitutes an additional hindrance to its cosmetic use as it is. Furthermore, the removal of chlorophylls is an operation which is complex on an industrial level.

### EXAMPLE 4

### Measurement of the Capacity of Absorption in the Visible and Ultraviolet Ranges of the Extracts of Example 3:

The extracts obtained are evaluated for their capacity of absorption in the visible and ultraviolet ranges on a Perkin Elmer Lambda 2 spectrophotometer, in quartz cuvettes, against ethanol.

It is noted that the extract of Leontopodium alpinum leaf (extract A, FIG. 1) exhibits a spectrum of absorption of UV-As, -Bs and -Cs from 200 to 400 nm with good continuity (outside 370 to 400 nm). Strong absorption is also found between 400 and 480 nm, as are weak absorptions above 570 nm. This absorption in the visible range is prejudicial to the industrial use of such an extract.

The absorption spectrum of the extract of dedifferentiated cells of Leontopodium alpinum (extract B, FIG. 2) covers UV-As, UV-Bs and UV-Cs up to approximately 360 nm.

The absorption spectrum of the extract of dedifferentiated cells of Leontopodium stracheyi (extract C, FIG. 3) covers only UV-Bs and -Cs.

It is therefore noted that the ethanol extracts of dedifferentiated cells of plants of the genus Leontopodium exhibit a capacity to absorb ultra-violet radiation and exhibit no absorption between 400 and 480 nm and no absorption above 570 nm, which makes them good candidates for industrial use as an agent for screening out ultraviolet radiation, and particularly in the cosmetics industry.

### EXAMPLE 5

Examples of compositions illustrating the invention. These compositions were obtained by simply mixing the various components. The amounts are indicated as a percentage of the total of the composition.

Composition 1:
Oil-in-water emulsion which screens out UV-As, -Bs and -Cs:

| | |
|---|---|
| Extract of dedifferentiated Leontopodium alpinum cells | 5.0 |
| Mixture of glyceryl mono/distearate/polyethylene glycol | 2.0 |
| stearate (100 EO) (ARLACEL 165 FL—ICI) | |
| Stearyl alcohol (LANETTE 18—HENKEL) | 1.0 |
| Stearic acid of palm oil (STEARINE TP— | 1.5 |
| STEARINERIE DUBOIS) | |
| Polydimethylsiloxane (DOW CORNING 200 FLUID— | 0.5 |
| DOW CORNING) | |
| Benzoate of C12/C15 alcohols (WITCONOL TN— | 5.0 |
| WITCO) | |
| Polydimethyl/(oxyethylenated-oxypropylenated | 1.0 |
| methylsiloxane) in solution at 10% in D5 (DC 5225 C— | |
| DOW CORNING) | |
| Polymethylphenylsiloxane (MIRASIL PTM— | 3.0 |
| RHODIA CHIMIE) | |
| Triethanolamine | 0.5 |
| Glycerol | 4.0 |
| Hexadecyl phosphate, potassium salt (AMPHISOL K— | 1.0 |
| HOFFMAN LAROCHE) | |
| Polyacrylic acid (SYNTHALEN K—3V) | 0.3 |
| Hydroxypropylmethylcellulose (METHOCEL F4M— | 0.1 |
| DOW CHEMICAL) | |
| Preserving agents | qs |
| Demineralized water | qs for 100.0% |
| Triethanolamine | qs pH = 7 |

Composition 2:
Water-in-oil emulsion which screens out UV-As, -Bs and -Cs:

| | |
|---|---|
| Extract of dedifferentiated Leontopodium alpinum cells | 1.5 |
| Polydimethyl/methylcetyl/(oxyethylenated | 2.0 |
| methylsiloxane) (ABIL EM 90D—GOLDSCHMIDT) | |
| Phenyltrimethylsiloxytrisiloxane (DOW CORNING 556 | 3.0 |
| COSMETIC grade fluid—DOW CORNING) | |
| Benzoate of C12/C15 alcohols (WITCONOL TN— | 8.0 |
| WITCO) | |
| Glycerol | 5.0 |
| Magnesium sulfate | 0.7 |
| Preserving agents | qs |
| Demineralized water | qs for 100.0% |

Composition 3:
Oil-in-water emulsion which screens out UV-Bs and -Cs:

| | |
|---|---|
| Extract of dedifferentiated Leontopodium stracheyi cells | 5.0 |
| Mixture of glyceryl mono/distearate/polyethylene glycol | 2.0 |
| stearate (100 EO) (ARLACEL 165 FL—ICI) | |
| Stearyl alcohol (LANETTE 18—HENKEL) | 1.0 |
| Stearic acid of palm oil (STEARINE TP— | 1.5 |
| STEARINERIE DUBOIS) | |
| Polydimethylsiloxane (DOW CORNING 200 FLUID— | 0.5 |
| DOW CORNING) | |
| Benzoate of C12/C15 alcohols (WITCONOL TN— | 5.0 |
| WITCO) | |
| Polydimethyl/(oxyethylenated-oxyproplenated | 1.0 |
| methylsiloxane) in solution at 10% in D5 (DC 5225 C— | |
| DOW CORNING) | |
| Polymethylphenylsiloxane (MIRASIL PTM— | 3.0 |
| RHODIA CHIMIE) | |
| Triethanolamine | 0.5 |
| Glycerol | 4.0 |
| Hexadecyl phosphate, potassium salt (AMPHISOL K— | 1.0 |
| HOFFMAN LAROCHE) | |
| Polyacrylic acid (SYNTHALEN K—3V) | 0.3 |
| Hydroxypropylmethylcellulose (METHOCEL F4M— | 0.1 |
| DOW CHEMICAL) | |
| Triethanolamine | qs pH = 7 |
| Preserving agents | qs |
| Demineralized water | qs for 100.0% |

Composition 4:
Water-in-oil emulsion which screens out UV-Bs and -Cs:

| | |
|---|---|
| Extract of Leontopodium stracheyi dedifferentiated cells | 1.5 |
| Polydimethyl/methylcetyl/(oxyethylenated | 2.0 |
| methylsiloxane) (ABIL EM 90D—GOLDSCHMIDT) | |
| Phenyltrimethylsiloxytrisiloxane (DOW CORNING 556 | 3.0 |
| COSMETIC grade fluid—DOW CORNING) | |
| Benzoate of C12/C15 alcohols (WITCONOL TN— | 8.0 |
| WITCO) | |
| Glycerol | 5.0 |
| Magnesium sulfate | 0.7 |
| Preserving agents | qs |
| Demineralized water | qs for 100.0% |

Composition 5:
Oil-in-water emulsion comprising several screening agents:

| | |
|---|---|
| Extract of dedifferentiated Leontopodium alpinum cells | 1.0 |
| Mixture of glyceryl mono/distearate/polyethylene glycol | 2.0 |
| stearate (100 EO) (ARLACEL 165 FL—ICI) | |
| Stearyl alcohol (LANETTE 18—HENKEL) | 1.0 |
| Stearic acid of palm oil (STEARINE TP— | 1.5 |
| STEARINERIE DUBOIS) | |
| Polydimethylsiloxane (DOW CORNING 200 FLUID— | 0.5 |
| DOW CORNING) | |
| Benzoate of C12/C15 alcohols (WITCONOL TN— | 5.0 |
| WITCO) | |
| Polydimethyl/(oxyethylenated-oxypropylenated | 1.0 |
| methylsiloxane) in solution at 10% in D5 (DC 5225 C— | |
| DOW CORNING) | |
| Polymethylphenylsiloxane (MIRASIL PTM— | 3.0 |
| RHODIA CHIMIE) | |
| Triethanolamine | 0.5 |
| 4-tert-Butyl-4′-methoxydibenzoylmethane | 1.0 |
| (PARSOL 1789—HOFFMANN LAROCHE) | |
| Octocrylene (UVINUL N539) | 8.0 |
| Titanium oxide (TITANIUM DIOXYDE MT-100 TV | 2.5 |
| TAYCA) | |
| Glycerol | 4.0 |
| 1,4-bis(benzimidazoylphenylene)-3,3′,5,5′- | 1.0 |
| tetrasulfonic acid—sodium salt | |
| Hexadecyl phosphate, potassium salt | 1.0 |
| (AMPHISOL K—HOFFMAN LAROCHE) | |
| Polyacrylic acid (SYNTHALEN K—3V) | 0.3 |
| Hydroxypropylmethylcellulose (METHOCEL F4M— | 0.1 |
| DOW CHEMICAL) | |
| Preserving agents | qs |
| Demineralized water | qs for 100.0% |
| Triethanolamine | qs pH = 7 |

Composition 6:
Water-in-oil emulsion comprising several screening agents:

| | |
|---|---|
| Extract of dedifferentiated Leontopodium alpinum cells | 0.5 |
| Polydimethyl/methylcetyl/(oxyethylenated | 2.0 |
| methylsiloxane) (ABIL EM 90D—GOLDSCHMIDT) | |
| Phenyltrimethylsiloxytrisiloxane (DOW CORNING 556 | 3.0 |
| COSMETIC grade fluid—DOW CORNING | |
| Benzoate of C12/C15 alcohols (WITCONOL TN— | 8.0 |
| WITCO) | |
| Methylenebis (tetramethylbutylhydroxyphenyl- | 5.0 |
| benzotriazole) | |
| Drometrizole trisiloxane | 2.0 |
| 2,4-bis{[4-(2-ethylhexyloxy)]-2-hydroxyphenyl}-6- | 2.0 |
| (4-methoxyphenyl)-1,3,5-triazine | |
| Titanium oxide (TITANIUM DIOXYDE MT100 TV— | 3.0 |
| TAYCA) | |
| Glycerol | 5.0 |
| Magnesium sulfate | 0.7 |
| Preserving agents | qs |
| Demineralized water | qs for 100.0% |

1. An extract of dedifferentiated cells of at least one plant of the genus Leontopodium.

2. The extract as claimed in claim 1, characterized in that the dedifferentiated cells are cells originating from a plant species chosen from the species Leontopodium alpinum, Leontopodium stracheyi, Leontopodium nivale, Leontopodium palibinianum (or Leontopodium sibiricum), Leontopodium souliei, Leontopodium linearifolium and Leontopodium gnaphalioides.

3. The extract as claimed in any one of the preceding claims, characterized in that the dedifferentiated cells are cells originating from a plant species chosen from the species Leontopodium alpinum and Leontopodium stracheyi.

4. The extract as claimed in any one of the preceding claims, characterized in that it is an alcoholic extract or an aqueous-alcoholic extract or an aqueous extract.

5. The extract as claimed in the preceding claim, characterized in that the alcoholic extract is an ethanol or methanol extract.

6. The extract as claimed in either one of claims 4 and 5, characterized in that the alcoholic extract is an ethanol extract.

7. An agent for screening out ultraviolet radiation, characterized in that it consists of at least one extract of dedifferentiated cells of at least one plant of the genus Leontopodium as defined in any one of the preceding claims.

8. An agent for screening out ultraviolet radiation type A, B and C, characterized in that it consists of at least one extract of dedifferentiated cells of at least one plant of the species Leontopodium alpinum .

9. An agent for screening out ultraviolet radiation type B and C, characterized in that it consists of at least one extract of dedifferentiated cells of at least one plant of the species Leontopodium stracheyi.

10. A composition comprising, in a physiologically acceptable medium, at least one extract of dedifferentiated cells of at least one plant of the genus Leontopodium as defined in any one of claims 1 to 6.

11. A composition for screening out ultra-violet radiation, comprising, in a physiologically acceptable medium, at least one agent for screening out ultraviolet radiation as defined in any one of claims 7 to 9.

12. A composition, characterized in that it comprises, in a physiologically acceptable medium, an extract of dedifferentiated cells of at least one plant of the genus Leontopodium and at least one other compound known as an agent for screening out ultra-violet radiation.

13. The composition as claimed in claim 12, characterized in that the agent for screening out ultraviolet radiation is chosen from organic screening agents and/or inorganic screening agents.

14. The composition as claimed in either one of claims 12 and 13, characterized in that the amount of agent for screening out ultraviolet radiation, other than the extract of dedifferentiated cells of at least one plant of the genus Leontopodium, is an amount representing from 0.1% to 20% of the total weight of the composition.

15. The composition as claimed in claim 14, characterized in that the amount of agent for screening out ultraviolet radiation of dedifferentiated cells of at least one plant of the genus Leontopodium is an amount representing from 0.5% to 10% of the total weight of the composition.

16. The composition as claimed in any one of claims 10 to 15, characterized in that the amount of extract of dedifferentiated cells of at least one plant of the genus Leontopodium contained in the composition is an amount representing from 0.001% to 10% of the total weight of the composition.

17. The,composition as claimed in claim 16, characterized in that the amount of extract of dedifferentiated cells of at least one plant of the genus Leontopodium contained in the composition is an amount representing from 0.1% to 5% of the total weight of the composition.

18. The composition as claimed in any one of claims 10 to 17, characterized in that it is intended for cosmetic use.

19. The use, in a composition or for preparing a composition, of at least one extract of dedifferentiated cells of at least one plant of the genus Leontopodium as defined in any one of claims 1 to 6, the extract or the composition being intended to screen out ultraviolet radiation.

20. The use, in a composition or for preparing a composition, of at least one screening agent as defined in any one of claims 7 to 9, the screening agent or the composition being intended to screen out ultraviolet radiation.

21. The use as claimed in any one of the preceding claims, for protecting the skin and/or the hair against ultraviolet radiation.

22. A method for the cosmetic treatment of the skin and/or the hair, intended to protect them against the effects of ultraviolet radiation, consisting in applying to the skin and/or to the hair an effective amount of a composition as defined in claim 18.
